(19) United States
(12) Patent Application Publication
(10) Pub. No.: US 2024/0255493 A1
(43) Pub. Date: Aug. 1, 2024

(54) CANCER SCREENING IN COMPANION ANIMALS

(71) Applicant: Oncotect, Inc., Raleigh, NC (US)

(72) Inventor: Chan Namgong, Raleigh, NC (US)

(21) Appl. No.: 18/585,626

(22) Filed: Feb. 23, 2024

Related U.S. Application Data
(63) Continuation-in-part of application No. PCT/US2022/041330, filed on Aug. 24, 2022.
(60) Provisional application No. 63/236,379, filed on Aug. 24, 2021.

(51) Int. Cl. G01N 33/50

(52) U.S. Cl. CPC G01N 33/5091; G01N 33/5085; G01N 2333/43534

(57) ABSTRACT

An economical and non-invasive method of using a behavioral response, a chemotaxis response, and/or a neuronal response of Caenorhabditis elegans (C. elegans) to screen for and/or detect cancer, such as screen for and/or detect early-stage cancer, in companion animals and humans is provided. Systems and kits using C. elegans to test and/or screen for and/or detect cancer, such as early-stage cancer, in companion animals and humans is also provided.

### CROSS-REFERENCE TO RELATED APPLICATIONS

This application is a bypass continuation-in-part application of International Application No. PCT/US2022/41330, filed Aug. 24, 2022, which claims priority to U.S. Provisional Application No. 63/236,379, filed Aug. 24, 2021, the contents of which are hereby incorporated herein as if set forth in their entireties.

### FIELD

The presently disclosed subject matter relates generally to screening for and/or detecting cancer in human and non-human animals, such as companion animals, and methods of monitoring the progression of cancer, determining the effect of cancer treatment and identifying therapeutic agents useful for treating cancer and kits for the same.

### BACKGROUND

There are over 200 million companion animals in the United States, and cancer is among the most common causes of death among them. 1 in 4 dogs will develop cancer, and almost half of all dogs over the age of ten will develop cancer. Cancer in companion animals is difficult to treat because few symptoms are evident in early-stage cancer. By the time symptoms become apparent, the cancer is usually too advanced to be treated. Early detection and treatment of cancer generally leads to more positive health outcomes. However, biopsy, ultrasound, and MRI screening options are often expensive and invasive, presenting a barrier to detecting early-stage cancers in companion animals. There is an urgent need to develop a novel economic and non-invasive method to routinely screen for cancer before it advances.

Although there have been several promising studies in which researchers have successfully screened human cancers using the acute olfactory sense of Caenorhabditis elegans (C. elegans), the results from these studies have yet to be translated into clinical success, and no notable commercial developments have been made in recent years. Moreover, relatively few studies have been conducted that examine the efficacy of using the olfactory sense of C. elegans to screen for cancers in non-human subjects, such as companion animals.

While cancer is a leading cause of death among canines, there remains a deficiency in the number of effective methods for cancer screening. Currently, tests such as the PetDx OncoK9 test are available on the market. These tests can detect a wide range of cancers that are present in canines. However, these are invasive tests which require the acquisition of a blood sample and can only be conducted in a veterinary clinic. Furthermore, at a cost of about $700 per test, these tests are frequently too expensive for many dog owners to use as a routine screen for the presence of cancer. The Cadet BRAF test is a non-invasive test; however, this test is limited to the detection of bladder and prostate cancer. Aside from these screening and diagnostic tests, cancer in companion animals, such as canines, is most often detected through medical procedures such as ultrasounds and MRI that are conducted in veterinary clinics. These procedures are generally non-invasive and allow for the type and location of cancer to be diagnosed. However, these procedures are expensive, and as a result are often only conducted when signs of cancer are already apparent. Early detection of cancer in companion animals, such as canines, can lead to treatment options that are more likely to be successful. Thus, there is a need in the veterinary field for a low-cost, non-invasive screening test for many different types of common cancers found in companion animals.

### SUMMARY

The present inventive concept provides non-invasive methods, kits, systems, and/or tests to screen for and/or detect cancer, and, according to some aspects, provides methods, kits, systems, and/or tests for screening and/or detection of early-stage cancer in a subject, wherein subjects include human and non-human animals, such as companion animals, for example, canines. The present inventive concept also provides methods to monitor cancer development as well as test for therapeutic agents to treat cancer. Non-invasive methods and/or tests of the present inventive concept include aspects of those described in Namgong et al. (2022) Front. Vet. Sci. 9, 932474, incorporated herein by reference.

Accordingly, in some aspects of the present inventive concept, provided is a method of screening for and/or detecting cancer in a subject, the method including detecting a behavioral, chemotaxis, and/or neuronal response of a nematode to a biological sample obtained from a companion animal wherein a positive response to the sample indicates the subject has cancer or is at risk for having cancer. In some aspects of the present inventive concept, the subject may be a companion animal, such as a canine. In some aspects of the present inventive concept, the nematode may be C. elegans. In some aspects of the present inventive concept, the C. elegans may be a wild-type, recombinant, or transgenic C. elegans. In some aspects of the present inventive concept, detecting of a behavioral and/or chemotaxis response of the nematode includes detecting if the response exceeds a threshold value for a positive response. In some aspects of the present inventive concept, detecting of a neuronal response of the nematode includes detecting of a repression of a neuronal response of the nematode, detecting of a lack of a neuronal response of the nematode, or detecting if the neuronal response of the nematode is within a threshold value for a positive response.

According to other aspects of the inventive concept, provided is a method of screening for and/or determining the effect of a treatment on a cancer in a subject, the method including detecting a behavioral, chemotaxis, and/or neuronal response of a nematode, or nematodes, to a biological sample obtained from a subject, wherein a positive response to the sample indicates the treatment has not rendered the cancer undetectable.

According to further aspects of the inventive concept, provided is a method of screening for and/or detecting cancer in a subject, the method including detecting a behavioral, chemotaxis, or neuronal response of a nematode, or nematodes, to (a) a biological sample obtained from a subject, and (b) at least one sample from the same or a different source, using a microfluidic approach to detect the chemotaxis response, wherein a predetermined response to the samples indicate the subject has cancer or is at risk for having cancer. In some aspects of the present inventive concept, the biological sample may be diluted prior to detecting the behavioral response, chemotaxis response, and/or neuronal response of the nematode to the biological sample.

According to further aspects of the inventive concept, provided is a kit for determining a presence of, and/or a risk of a presence of a cancer in a subject; or determining an effect of a treatment on cancer development in subject, wherein the kit includes: a receptable to contain a biological sample from the subject; and instructions providing guidance regarding the biological sample and steps to obtain results from a test to determine a behavioral response, a chemotaxis response, and/or a neuronal response of a nematode to the biological sample.

According to further aspects of the inventive concept, provided is a computer-implemented method executed on a computer system, or computer readable medium operated thereon for: (i) determining a presence, and/or a risk of a presence of a cancer in a subject; or (ii) determining an effect of a treatment on cancer development in subject, the method including: monitoring a behavioral response, a chemotaxis response or a neuronal response for a nematode, or a plurality of nematodes, to a biological sample; and determining whether the behavioral response, chemotaxis response or the repression of a neuronal response exceeds or is within a threshold for a positive response, wherein the presence of cancer in the subject is determined if the chemotaxis response or the repression of neuronal response exceeds the threshold for a positive response. In some aspects, the determining of whether the behavioral response, chemotaxis response and/or repression of the neuronal response exceeds or is within a threshold for a positive response includes evaluation of the behavioral response, chemotaxis response or the repression of neuronal response of the nematode or plurality of nematodes via analysis using artificial intelligence and/or machine learning. In some aspects of the present inventive concept, the determining of whether cancer is present, and/or a risk of cancer is present in the subject further includes uranalysis and GC/MS analysis of the biological sample.

### BRIEF DESCRIPTION OF THE DRAWINGS

Having thus described the presently disclosed subject matter in general terms, reference will now be made to the accompanying drawings, which are not necessarily drawn to scale, and wherein:

FIG. 1 illustrates chemotaxis of C. elegans toward urine from canine cancer patients. Shown here are two different chemotaxis assay plate images, one without cancer urine samples (left) and one with cancer cells (right).

FIG. 2 depicts an overview of the on-plate Animal Cancer Detection (A.C.D.) Test.

From Namgong et al. (2022) Front. Vet. Sci. 9, 932474.

FIG. 3 depicts (panel A) Mean CI plotted for eight cancer and 14 non-cancer samples for which the A.C.D. Test was conducted. A mean CI of 0.099±0.038 for cancer samples versus a mean CI of −0.006±0.032 in non-cancer samples (p=0.0002) Red line indicates moderate to high cancer risk classification threshold. **P<0.01 ***p<0.001 (panel B) Levels of cancer risk set at the following range: Low Risk (<˜0.038) and Moderate to High Risk (>˜0.038)

FIG. 4 provides an exemplary cancer risk assessment scale provided by a diagnostic test of the present inventive concept.

FIG. 5 depicts a scaled-down design for an on-chip test platform for detecting AWC response to urine odorant stimuli. Worms are loaded and unloaded from a single channel, while each sample is fed into a separate chamber. Neuronal response of individual worms is recorded in the imaging chamber. A porous barrier on the stimulus outlet prevents worms from exiting the chamber during assays.

FIG. 6 depicts a flowchart illustrating included components and analysis conducted by an exemplary system of the inventive concept.

### DETAILED DESCRIPTION

The presently disclosed subject matter now will be described more fully hereinafter with reference to the accompanying drawings, in which some, but not all embodiments of the presently disclosed subject matter are shown. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth. By way of example, “an element” means at least one element and can include more than one element. The term “and/or” includes any and all combinations of one, or more, of the associated listed items and may be abbreviated as “/”. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. Expressions such as “at least one of,” when preceding a list of elements, modify the entire list of elements and do not modify the individual elements of the list.

Throughout this specification and the claims, the terms “comprise,” “comprises,” and “comprising” are used in a non-exclusive sense, except where the context requires otherwise. Likewise, the term “include” and its grammatical variants are intended to be non-limiting, or “open” terms, such that recitation of items in a list is not to the exclusion of other like items that can be substituted or added to the listed items (e.g., the term “including” should be interpreted as “including but not limited to,” the term “having” should be interpreted as “having at least,” the term “includes” should be interpreted as “includes but is not limited to,” etc.).

It will also be appreciated that the term “comprise,” as used herein, may also encompass, and, in some embodiments, may specifically refer to the expressions “consist essentially of” and/or “consist of.” Thus, the expression “comprise” can also refer to embodiments, wherein that which is claimed “comprises” specifically listed elements does not include further elements, as well as embodiments wherein that which is claimed “comprises” specifically listed elements may and/or does encompass further elements, or may and/or does encompass further elements that do not materially affect the basic and novel characteristic(s) of that which is claimed. For example, that which is claimed, such as a method, kit, system, etc. “comprising” specifically listed elements also encompasses, for example, a method, kit, system, etc. “consisting of,” i.e., wherein that which is claimed does not encompass any further elements, and, for example, a method, kit, system, etc. “consisting essentially of,” i.e., wherein that which is claimed may include further elements that do not materially affect the basic and novel characteristic(s) of that which is claimed. Similarly, it will be appreciated that other open terms (include, including, have, having, etc.) may refer to and encompass the specifically listed elements, but are not limited to (comprise, comprising, etc.), encompass the specifically listed elements only without any further elements (consists of, consisting of, etc.), and encompass the specifically listed elements, with further elements that do not materially affect the basic and novel characteristic(s) set forth as claimed (consists essentially of, consisting essentially of, etc.).

The abbreviation, “e.g.,” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

The term “about,” when referring to a value can be meant to encompass variations of, in some embodiments ±100%, in some embodiments ±50%, in some embodiments ±20%, in some embodiments ±15%, in some embodiments ±10%, in some embodiments ±5%, ±4%, ±3%, or ±2%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The term “screening” or “screening for” a disease/disorder, e.g., a cancer, as used herein may include, for example, routine testing of patients/subjects, either with symptoms or without symptoms, for an abnormality/disease/disorder, such as a cancer. Further testing may be included for the patient/subject if the screening indicates the potential for/the probability for a presence of an abnormality/disease/disorder, such as a cancer, in the subject. The term “diagnostic,” “diagnosing,” or “diagnosis for,” as used herein, may include testing to investigate symptoms that a subject/patient may have, or testing to investigate the potential presence of an abnormality/disease/disorder that may have been detected through identifying, screening, and/or detecting the presence of an abnormality/disease/disorder in the subject.

The present inventive concept relies, in part, on using a nematode, i.e., Caenorhabditis elegans (or C. elegans). As a primitive organism. C. elegans is often used to study neural development in more complex animals. Its genome was the first to be sequenced due to its simplicity. In nature, C. elegans live in soil. Because it has no eyes and cars, it relies on smell to navigate its environment and detect food.

In some embodiments of the present inventive concept, a method of screening for and/or detecting cancer in a subject, such as a companion animal is provided. The method includes detecting a behavioral response, a chemotaxis response, and/or a neuronal response of a nematode, or behavioral response, a chemotaxis response, or neuronal response of more than one nematode, e.g., the response to a plurality of nematodes, to a biological sample obtained from a subject, such as a companion animal, wherein a positive response to the sample indicates the subject has a cancer or is at risk for having a cancer. In some embodiments of the present inventive concept, the method of screening for and/or detecting cancer is included in methods of treating a subject, wherein the subject is treated for cancer if the subject is determined to have a cancer or is at risk for having a cancer by the methods of screening for and/or detecting cancer as described herein. In some embodiments, the screening for and/or detection of a cancer may include early detection and/or early diagnosis (downstaging) of the cancer, for example, detection of the cancer prior to metastasis, detection of cancer at or shortly after symptom onset, detection of preinvasive cancer, and/or detection of abnormal/precancerous cells in the subject.

As used herein, a “positive response” or a “positive chemotaxis response” of, for example, a nematode or nematodes to a sample, indicating a subject has a cancer or is at risk of having a cancer, can be understood as the nematode “is not interested in” and/or “moves away from/does not move toward” a sample. Accordingly, when a chemotaxis response is used as an indicator, a positive response may include chemotaxis toward the sample, and/or a chemotaxis response exceeding a threshold level of chemotaxis to the sample by the nematode and/or nematodes, and a negative response may include chemotaxis away from, a lack of chemotaxis toward, and/or a chemotaxis response not exceeding a threshold level of response to the sample by the nematode and/or nematodes. Similarly, in embodiments wherein neuronal activity/neuronal response is used as an indicator, the repression of neuronal activity/response, the lack of neuronal activity/response, or the presence of neuronal activity/response that is within a threshold level of neuronal response/activity can be understood as being a positive response by the nematode and/or nematodes, whereas the presence of neuronal activity that is above a threshold level can be understood as being a negative response by the nematode and/or nematodes.

In embodiments wherein more than one nematode, e.g., a plurality of nematodes, is used in determining a chemotaxis response or a neuronal response, the number of nematodes used is not particularly limited. The number of nematodes used may be, for example, about 2, 3, 4, 5, 6, 7, 8, 9, about 10, about 15, about 20, about 25, or about 30 nematodes, or in about 2-30 nematodes, or even more than about 30 nematodes, for example, about 40 nematodes, about 50 nematodes, about 60 nematodes, about 70 nematodes, about 80 nematodes, about 90 nematodes, about 100 nematodes, about 150 nematodes, about 200 nematodes, about 250 nematodes, about 300 nematodes, about 350 nematodes, about 400 nematodes, about 450 nematodes, about 500 nematodes, about 600 nematodes, about 700 nematodes, about 800 nematodes, or about 900 nematodes, may be used in determining a chemotaxis response or a neuronal response. Use of more than one nematode, and/or use of more than one nematode of about the same size and/or age, in the determination of a chemotaxis and/or neuronal response to the sample may be useful in reducing the number of false positive and/or false negative responses/results as determined by the methods of the present inventive concept. In some embodiments, data acquisition may include discarding of tests according to the conditions as described in Namgong et al. (2022) Front. Vet. Sci. 9, 932474.

As used herein, subjects of the present inventive concept include human and non-human animals. Humans may be any gender or gender identity, including, but not limited to, female, male and transgendered individuals. Human subjects may be any age such as less than 12 months to over 100 years including newborns, infants, juveniles, adolescents, teenagers, adults and geriatrics. Further human subjects may be of any race or ethnicity, including, but not limited to, Caucasian, African American, African, Asian, Hispanic, South Asian, etc., and combined/mixed backgrounds.

Non-human animals include, e.g., mammals, such as canines, felines, bovines, caprines, equines, ovines, porcines, rodents (e.g., rats and mice), lagomorphs, and primates (including non-human primates). In some embodiments, non-human animals include avians. Non-human animals may also be domesticated animals, companion animals and/or wild animals for veterinary medicine, treatment or pharmaceutical drug development purposes. “Domesticated animal” as used herein refers to any species customarily maintained in domestication in any environment or ethnic culture including, but not limited to cows, buffalo, camels, sheep, goats, horses, poultry, swine and llamas. In some embodiments, the animal is a companion animal. “Companion animal” as used herein refers to the species of animals kept by humans as pets or for benefit and/or enjoyment and are not typically for food production. Companion animals, include, but are not limited to, an animal such as a dog, cat, horse, goat, rabbit, pig, ferret, guinea pig, hamster, gerbil or bird, and the like. Embodiments of the present inventive concept include methods, systems, and/or kits for cancer screening and/or detection, in some embodiments, early cancer detection, in a subject/animal, in some embodiments, companion animals, and treatment of a subject/animal determined as having a cancer using the methods, systems, and/or kits of the present inventive concept.

According to some embodiments, the subject can also be one who has been previously diagnosed with or identified as suffering from or having a condition in need of treatment (e.g., a cancer) or one or more complications related to the condition, and optionally, have already undergone treatment for the condition or the one or more complications related to the condition. Alternatively, the subject can also be one who has not been previously diagnosed as having a condition or one or more complications related to the condition. For example, the subject can be one who exhibits one or more risk factors for a condition, or one or more complications related to the condition or a subject who does not exhibit risk factors. A “subject in need” of treatment for a particular condition can be a subject suspected of having that condition, diagnosed as having that condition, is being treated or has already been treated for the particular condition, and if not being treated for the particular condition, is considered or is at risk of developing the particular condition.

The term “sample” or “biological sample,” as used herein, may denote a sample derived from a subject or subjects to be used in the methods of the present inventive concept. Exemplary biological samples include, but are not limited to, tissues, organs, cells and bodily fluids. In some embodiments, the sample is a bodily fluid, for example, urine, blood (whole blood or any derivative thereof, serum, plasma, clotted blood, dried blood, etc.), mucus, saliva, tears, etc. The term “sample” also includes untreated or pretreated (or pre-processed) biological samples. In some embodiments, a sample may include one or more cells from the subject.

In some embodiments, the pretreated or pre-processed sample may include dilution of the sample in a diluent/diluting liquid, for example, such as water, a buffer, a saline solution, e.g., normal and/or physiological saline, or an odor-neutral control buffer, such as CTX buffer (5 mM KH₂PO₄/K₂HPO₄ pH 6, 1 mM CaCl₂) and 1 mM MgSO₄), and the like, prior to determining the chemotaxis response of a nematode to the sample/biological sample. The dilution factor is not particularly limited, and may be in a range of about 2:1-1:10,000,000,000, for example, about 2:1, about 1:1, about 1:1.5, about 1:2, about 1:3, about 1:4, about 1:5, about 1:6, about 1:7, about 1:8, about 1:9, about 1:10, about 1:15, about 1:20, about 1:25, about 1:30, about 1:40, about 1:50, about 1:75, about 1:100, about 1:200, about 1:500, about 1:1,000, about 1:5,000, about 1:10,000, about 1:100,000 (10⁵), about 1:1,000,000 (10⁶), about 1:10,000,000 (10⁷), about 1:100,000,000 (10⁸), about 1:1,000,000,000 (10⁹), or even about 1:10,000,000,000 (10¹⁰), sample to diluent prior to determining the chemotaxis response of the nematode. In some embodiments, the range of the ratio of dilution may be about 1:1-1:10⁹, about 1:1-1:10⁸, about 1:1-1:10⁷, about 1:1-1:10⁶, about 1:1-1:10⁵, about 1:1-1:104, about 1:1-1:103, or about 1:1-1:102. In some embodiments, the range of the dilution factor may be between any combination of more particular dilutions listed above without limitation.

The nematode used in methods, systems, kits, and/or tests of the present inventive concept can be a wild-type nematode, a mutant nematode a recombinant nematode, and/or a transgenic nematode. In some embodiments, the nematode is Caenorhabditis elegans (C. elegans). In some embodiments, the nematodes used in the methods, systems, kits, and/or tests of the present inventive concept may be synchronized based on size and/or stage of development, e.g., as described by Porta-de-la-Riva et al. J. Vis. Exp. (2012) 64, e4019, incorporated herein by reference. In some embodiments, the stage of development of the nematode, or nematodes, used in the present inventive concept are, for example, young adult, non-gravid (non-egg-laying) C. elegans. It will be appreciated by one of skill in the art that a typical size of a young adult, non-gravid C. elegans is about 1-1.2 mm in length, are typically transparent, and/or can be observed under a microscope. It will also be appreciated that a nematode used in methods according to the present inventive concept, such as C. elegans, has an acute sense of smell—with at least 1.5-fold greater number of types of olfactory receptors than a dog. In some embodiments, the nematode used is a mutant C. elegans a recombinant C. elegans, and/or a transgenic C. elegans, such as the transgenic C. elegans strains as described in in U.S. Patent Application Publication No. 2017/0016906, incorporated herein by reference.

It will be appreciated by one of skill in the art that once a nematode, such as C. elegans, detects, for example, an odorant/chemical that it is attracted to, it aligns with the chemical odorant and travels toward the odorant in a process known as chemotaxis. This acute sense of smell allows for C. elegans to detect distinct volatile organic compound (VOC), or volatile organic metabolite (VOM), profiles within animal urine. The “volatilome” is the collection of VOCs/VOMs which are present in the output from cells of the biological organism. It will also be appreciated by one of skill in the art that cancer cells emit VOCs that produce an odor that is distinguishable from that of non-cancer patients. Accordingly, by utilizing the acute sense of smell of C. elegans, detection of VOCs characteristic of cancer cells, and thus detection of the presence of cancer in a subject is possible based on the difference between the behavioral response, e.g., chemotaxis response, and/or neuronal response, of C. elegans to a biological sample derived from a subject having cancer/cancer cells present, as opposed to the behavioral/chemotaxis response and/or neuronal response of C. elegans to a biological sample derived from a subject that is free of cancer.

The VOC/VOM, or VOCs/VOMs, characteristic of cancer cells that are detected by the methods, systems, kits, and/or tests of the present inventive concept is not particularly limited, and it will be appreciated by one of skill in the art that the VOC/VOM or VOCs/VOMs detected may be any that has been determined to be characteristic of cancer cells, and, in some embodiments, a VOC/VOM, or VOCs/VOMs, to which a nematode, or nematodes, exhibit a positive behavioral response, chemotaxis response, and/or neuronal response, such as, but not limited to those described in, e.g., Opitz and Herbarth. (2018) J. Otolaryngol.—Head Neck Surg. 47, 1-13, incorporated herein by reference. Exemplary VOCs/VOMs include, but are not limited to, for example, diacetyl, propyl acetate, butyl acetate, hexyl acetate, isoamyl alcohol, isopropanol pyrazine, 2-methylpyrazine, 2,4,5-trimethyl thiazole, 2,3-pentadione, 1,2,4-trimthylbenzene, 1,4-xylene, 1-methyl-4-propan-2-ylbenzene, and 2-ethylhexan-1-ol, and the like.

The nematode used in the methods of the present inventive concept may include either male or female nematodes. However, in some embodiments, the nematode may be a hermaphrodite that can self-proliferate. The nematode used in the methods of the present inventive concept may be readily bred, for example, in a petri dish and fed with Escherichia coli. If a parent nematode is transferred into a petri dish, larvae are born and can typically be grown to adult worms in about four days, while increasing the number of nematodes in this time by about 50-100-fold. Breeding of the nematodes requires no special operations, and in the case where a hermaphrodite nematode is used, mating is unnecessary. The basic requirements for breeding of nematodes, such as C. elegans, are minimal and include a 20° C. incubator and a stereoscopic microscope, and thus, the nematodes required for the methods of the present inventive concept can be established in a short period of time at nominal costs.

An exemplary wild-type nematode that may be used in the methods of the present inventive concept is C. elegans Bristol N2, which includes, in some embodiments, the hermaphrodite of this C. elegans strain. Nevertheless, mutant strains of C. elegans may be used in the methods of the present inventive concept, such as any appropriate mutant as may be available through e.g., the Caenorhabditis Genetic Center (CGC) at the University of Minnesota (<ege.umn.edu>), and/or as may be available as a courtesy through, for example, any other lab without limitation.

In some embodiments of the present inventive concept, in addition to the aforementioned wild-type nematodes and mutant nematodes discussed above, transgenic nematodes, i.e., a nematode in which a foreign gene has been introduced, may also be used in the methods of the present inventive concept. Exemplary transgenic nematodes include, but are not limit to, for example: a nematode in which an indicator/reporter gene has been introduced into one of the olfactory neurons, e.g., the AWC and/or AWA neuron(s); a nematode, in which the expression or function of a gene associated with a receptor associated with smelling/sensing of cancer has been inhibited; a nematode, in which a gene associated with a receptor associated with smelling/sensing of VOCs characteristic of cancer has been overexpressed; and a nematode in which a fluorescent reporter protein is expressed that facilitates analysis of the behavior, e.g., chemotaxis and/or chemical/neuronal response of the nematode.

Analysis of behavior, e.g., analysis of chemosensation, including chemotaxis and chemoaversion behaviors, of a nematode, or nematodes, including wild-type, mutant, and/or transgenic strains of C. elegans, may be conducted, for example, as described in WormBook <www.wormbook.org/chapters/www_behavior/behavior.html>, incorporated herein by reference. Analysis of neuronal response of a nematode, or nematodes, may be conducted for example, by measuring/monitoring changes in calcium concentrations and/or calcium response in neurons of the nematode or nematodes.

In some embodiments, screening for and/or detecting of cancer using the methods, systems, and/or kits of the present inventive concept may include use of a transgenic nematode, in which an indicator gene capable of measuring/monitoring calcium concentration in a neuron is expressed in an olfactory neuron(s), for example, AWC and/or AWA, of the nematode, is used, and presence of cancer may be detected by measuring/monitoring changes in calcium concentration (neuronal response) as an indicator, for example, as described by Chronis et al. (2007) Nat. Methods 4, 727-731, and Larsch et al. (2013) Proc. Natl. Acad. Sci. USA 110, E4266-E4273, incorporated herein by reference, and in some embodiments, using a transgenic nematode, for example, as described in U.S. Application Publication No. 2017/0016906.

According to some embodiments of the present inventive concept, the positive response exhibited by the nematode, or nematodes, used in the methods, systems, kits, and/or tests of the present inventive concept as described herein is a chemotaxis response that includes chemotaxis of the nematode, or nematodes, toward the biological sample.

Cancer screened for and/or detected in a subject, such as a companion animal, by the methods, systems, kits, and/or tests of the present inventive concept include all cancers and tumors, for example, but not limited to, breast cancer, liver cancer, kidney cancer, pancreatic cancer, thyroid cancer, lung cancer, esophageal cancer, head and neck cancer, colon cancer, rectal cancer, colorectal cancer, gastric cancer, intestinal cancer, gastrointestinal cancer, cervical cancer, uterine cancer, ovarian cancer, bladder cancer, prostate cancer, skin cancer, brain cancer, and any metastases of any thereof. In some embodiments, the cancer is hemangioma, lymphoma, osteosarcoma, plasma cell tumor, and mast cell tumor, transitional cell carcinoma, squamous cell carcinoma, fibrosarcoma, mammary (breast) carcinoma, and/or melanoma in companion animals, i.e., common cancers that afflict companion animals, such as, but not limited to, dogs and/or cats.

Embodiments of the present inventive concept further provide methods for determining the effect of a treatment on the development of cancer in a subject, such as a companion animal. The methods include detecting a chemotaxis response of a nematode to a biological sample obtained from a companion animal wherein a positive response to the sample indicates the treatment has not rendered the cancer undetectable. More specifically, this method allows an animal caregiver to assess whether treatment being administered to an animal with cancer is having an effect of treating the cancer. Likewise, the method can be used to determine whether a specific compound is useful for the treatment of cancer in an animal.

Embodiments of the present inventive concept also provide kits for determining the presence of cancer in an animal as well as determining the effect of a treatment on development of cancer in an animal as well as determining whether a specific compound is useful for the treatment of cancer in an animal.

Embodiments of the present inventive concept further reduce the incidence of false positive and/or false negative cases. In particular, by determining the chemotaxis response or chemotaxis index in at least four (4) replicates of an assay for chemotaxis response to determine an average chemotaxis index, the accuracy of cancer detection and determination of the effect of cancer treatment on cancer development and/or discovery of a cancer agent for the subject can be improved.

In some embodiments, the Student t-test is used to determine whether chemotaxis was significant. Mean chemotaxis of C. elegans can be employed to determine if there were positive chemotaxis toward the cancer urine samples (or non-cancer control samples)—towards the plus signs—or negative chemotaxis toward the minus signs. The mean difference is compared to determine whether it was positive (comparing non-cancer to non-cancer samples) or negative (comparing non-cancer to positive samples). Finally, a positive pattern test is conducted to measure the percent of positive chemotaxis patterns. Based on the at least 4 replicates of an assay for chemotaxis response to determine an average chemotaxis index, the results were combined to render an assessment of the subject's cancer risk and/or efficacy of treatment with an agent. An assessment may then be made whether the subject has a cancer or is at risk for having a cancer if a threshold for a positive response is exceeded by, for example, the behavioral response, chemotaxis response, and/or repression of the neuronal response. Accordingly, in some embodiments of the present inventive concept, a determination of a positive response may identify and/or classify a subject as having cancer and/or as more likely than not as having cancer, and a determination of a negative response may identify and/or classify the subject as not having cancer and/or as less likely to have cancer. In some embodiments, the determination of a positive response may identify and/or classify treatment of a subject with cancer has not rendered the cancer undetectable, and a determination of a negative response may identify and/or classify the treatment of the subject with cancer has rendered the cancer undetectable.

The threshold for a positive response is not particularly limited, and may be determined based on the behavioral response, chemotaxis response, and/or neuronal response that is being used in an assay. For example, when measuring a chemotaxis response of a nematode or nematodes, the threshold for average chemotaxis index (CI) may be in a range of about 0.01-0.06, about 0.02-0.05, about 0.02-0.04, or about 0.03-0.04. In some embodiments, the threshold may be about 0.038-0.04. In some embodiments, an average or mean CI value below or less than or equal to the threshold for a positive response may classify/identify the subject as being at “low risk” for cancer. In some embodiments, an average or mean CI value above or greater than the threshold for a positive response may classify/identify the subject as being at “moderate to high risk” for cancer. In some embodiments, an average or mean CI value of about or greater than about 0.1 may further classify/identify the subject as being at “high risk” for cancer. In some embodiments, when measuring a neuronal response, such as a calcium response of an AWC and/or AWA neuron of a nematode is used, the subject may be classified/determined/identified to have a cancer or is at risk for having a cancer if the calcium response is within a threshold for normalized fluorescence intensity. In some embodiments, the threshold for normalized fluorescence intensity is in a range, for example, of about 1.5-4 times the baseline fluorescence activity. In some embodiments, the threshold for normalized fluorescence intensity is in a range of about 2-3 times the baseline fluorescence activity.

Embodiments of the inventive concept may also include use of artificial intelligence (AI) and/or machine learning (ML) to accumulate and analyze data related to the response, and/or analyze data related to the significance of the response, in the nematode or nematodes used in the methods, systems, and/or kits of the inventive concept to classify/diagnose/determine/identify if the subject has or may have a cancer or is at risk of having a cancer, and/or prescribe/determine a course of treatment for the subject.

It will be appreciated by one of skill in the art, AI and/or ML relates to the study and use of computer algorithms that improve automatically through experience and by the use/accumulation of data. AI and ML algorithms may build a model based on sample data (known as “training data”) in order to make predictions or decisions without being explicitly programmed to do so. Artificial intelligence and machine learning algorithms may be used in a wide variety of applications, such as in medicine, for example, in medical diagnosis and treatment, such as the methods of determining and treating as described herein, as well as in a broad range of applications such as email filtering, speech recognition, and computer vision, or any application wherein it may be difficult or unfeasible to develop conventional algorithms to perform the needed tasks. AI and ML may involve computers discovering how they can perform tasks without being explicitly programmed to do so (e.g., where computers learn from sample data (known as “training data”) how to carry out certain tasks.

It will also be appreciated by one of skill in the art that a machine learning system or model may generally include an algorithm (or combination of algorithms) that has been trained to recognize certain types of patterns. For example, machine learning approaches may be generally divided into three categories, depending on the nature of the signal available: supervised learning, unsupervised learning, and reinforcement learning. Supervised learning may include presenting a computing device with example inputs and their desired outputs, given by a “teacher”, where the goal is to learn a general rule that maps inputs to outputs. With unsupervised learning, no labels are given to the learning algorithm, leaving it on its own to find structure in its input. Unsupervised learning can be a goal in itself (discovering hidden patterns in data) or a means towards an end (feature learning). Reinforcement learning may generally include a computing device interacting in a dynamic environment in which it must perform a certain goal (such as driving a vehicle or playing a game against an opponent). As it navigates its problem space, the program is provided feedback that is analogous to rewards, which it tries to maximize. Nevertheless, it will be appreciated that other machine learning approaches are possible within the scope of the present disclosure.

Accordingly, embodiments of the present inventive concept may include analysis of behavioral responses, e.g., chemotaxis, chemoaversion, and/or odortaxis responses, and/or neuronal activity, such as repression and/or lack of neuronal activity, of a nematode or nematodes determined using the methods, systems and/or kits of the present inventive concept, and determining if any of these responses exceed a threshold value for a positive response as described herein, may be performed using/with the assistance of AI and/or ML to arrive at a diagnosis/determination of whether the subject has a cancer, or a subject is at risk for developing a cancer. Similarly, additional analyses used in screening for a cancer in a subject/patient, diagnosis/determination of whether a subject/patient has a cancer, and/or assessing whether a subject is at risk of developing a cancer may be performed in conjunction with the embodiments of the present inventive concept, such as urinalysis, e.g., chemical evaluation of the sample using urine test strips, measurement of chemical properties of the sample (e.g., pH, glucose concentration, protein levels, etc.), microscopic examination of the sample, and/or GC/MS analysis of the sample, as well as use of additional information that may be obtained from the subject/patient, such as, e.g., age, gender, medical history, and the like, may be utilized in order to arrive at an risk assessment/determination whether the subject/patient has cancer using/with the assistance of AI and/or ML, without departing from the scope of the present inventive concept. These embodiments may also include, as will be appreciated by one of skill in the art, methods, systems, and/or kits including, for example, a computer program product and/or computer-implemented methods that facilitate performing the methods of determining and/or treating as described herein.

Any suitable computer and/or computer system, for example, a personal computer, a server computer, a series of server computers, a mini computer, a mainframe computer, one or more Network Attached Storage (NAS) systems, one or more Storage Area Network (SAN) systems, one or more Platform as a Service (PaaS) systems, one or more Infrastructure as a Service (IaaS) systems, one or more Software as a Service (SaaS) systems, a cloud-based computational system, and a cloud-based storage platform, or computer readable medium included and/or operated thereon, may be utilized to run the computer program products and/or execute the computer-implemented methods of the present inventive concept. The computer-usable or computer-readable medium may be, for example but not limited to, an electronic, magnetic, optical, electromagnetic, infrared, or semiconductor system, apparatus, device, or propagation medium. More examples of the computer-readable medium may include, but are not limited to, the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a transmission media such as those supporting the Internet or an intranet, or a magnetic storage device. The computer-usable or computer-readable medium may also be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-usable or computer-readable medium may be any medium that can contain, store, communicate, propagate, or transport the program for use by or in connection with the instruction execution system, apparatus, or device. The computer-usable medium may include a propagated data signal with the computer-usable program code embodied therewith, either in baseband or as part of a carrier wave. The computer usable program code may be transmitted using any appropriate medium including, but not limited to, the Internet, wireline, optical fiber cable, RF, etc.

Computer program code for carrying out operations of the present disclosure may be written in an object-oriented programming language such as, but not limited to, Java, Smalltalk, C++ or the like. However, the computer program code for carrying out operations of the present disclosure may also be written in conventional procedural programming languages, such as the “C” programming language or similar programming languages. The program code may execute entirely on the computer of the user, partly on the computer of the user, as a stand-alone software package, partly on the computer of the user and partly on a remote computer or entirely on the remote computer or server. In the latter scenario, the remote computer may be connected to the computer of the user through a local area network (LAN)/a wide area network (WAN)/the Internet.

Embodiments of the present inventive concept also provide a microfluidic approach as compared to a plate-based assay approach for early cancer detection in subjects including human and non-human animals. The microfluidic method allows highly quantitative behavioral, chemotaxis, odortaxis, and/or neuronal activity phenotypic assays by increasing experimental control and throughput. In many cases, the microfluidic method only requires one C. elegans to run each assay, takes less than 30 mins to run each assay from preparation to data analysis, and it is a high-throughput process. Exemplary microfluidic methods of determining chemotaxis and/or neuronal activity are described in, e.g., by Chronis et al. (2007) Nat. Methods 4, 727-731, and Larsch et al. (2013) Proc. Natl. Acad. Sci. USA 110, E4266-E4273.

Methods, systems, kits, and/or tests of the present inventive concept for diagnosis/determination/identification of whether a subject has a cancer, or a subject is at risk for developing a cancer may also include: age-synchronization of nematodes by size using mechanical and/or visual filtration; automated, sequential analysis of samples, e.g., urine samples that have been inputted by the user; operational software including an associated graphic user interface (GUI) that facilitates straightforward user operation; automated preparation/pretreatment of samples, e.g., automated sample/urine dilution with a control buffer/diluting liquid; methods, systems, and/or kits that facilitate chemotaxis analysis of nematodes toward samples conducted simultaneously with calcium/neuronal response measurements; automated identification and tracking of a nematode or nematodes; AI-based algorithms for tracking behavior, e.g., chemotaxis/movement/chemoaversion, and/or measuring calcium response of individual worms/nematodes to facilitate determination of response over time; and AI-based classification of cancer risk based on all acquired data including, for example, but not limited to, urinalysis of the sample, and/or GC/MS analysis of the sample, and/or medical history of the subject, may assist in and/or supplement the classification of whether the subject/patient has, and/or is at risk for developing cancer, in addition the use of AI-based classification in evaluation of behavioral, chemotaxis, and/or neuronal responses of the present inventive concept as described herein to provide a comprehensive cancer assessment for the subject/patient. The urinalysis and/or GC/MS analysis methods used to assist with and/or to supplement the classification are not particularly limited, and may be any protocol for urinalysis and/or GC/MS analysis as would be appreciated by one of skill in the art.

Further aspects and advantages of the present inventive concept are disclosed in the following experimental section, which illustrates the present inventive concept without imposing any limitation thereto.

### Examples

### 1. Early Cancer Screening/Detection-Methods and Results

A novel cancer screening/detection system for companion animals was developed and tested for a variety of characteristics. It was found to perform with about 85% sensitivity and about 90% specificity. These methods and results make up in part that which is set forth in Namgong et al. (2022) Front. Vet. Sci. 9, 932474, as well as the Supplementary Material included therewith (<www.frontiersin.org/articles/10.3389/fvets.2022.932474/full#supplementary material>), the disclosures of which are incorporated herein by reference.

### Canine Urine Samples

All samples from non-cancer patients, as well as an initial set of samples from cancer patients were obtained from Triangle Veterinary Hospital (Durham, NC), Lake Pine Animal Hospital (Apex, NC), Care First Animal Hospital (Raleigh, NC) New Light Animal Hospital (Wake Forest, NC), Bull City Veterinary Hospital (Durham, NC), Knightdale Animal Hospital (Knightdale, NC). The second set of 40 cancer samples (10 samples each from patients with lymphoma, mast cell tumor, melanoma, and hemangiosarcoma) was obtained from the Ohio State University Center for Clinical and Translational Science. Upon acquisition, urine samples were immediately stored at −20° C. until assays were conducted. Each specimen was aliquoted into 100 μL portions to minimize repeat freezing and thawing each time an assay is performed.

Maintenance of C. elegans

Caenorhabditis elegans strain N2 and Escherichia coli strain HB101 were obtained from the Caenorhabditis Genetics Center (University of Minnesota). C. elegans were age-synchronized by standard bleaching protocols and was cultured at 20° C. on nematode growth media (NGM) plates seeded with HB101 bacterial lawns. NGM plates were purchased pre-poured from LabExpress (Ann Arbor, MI).

### Chemotaxis Assays

Assays are performed using well-fed age-synchronized populations of N2 worms grown at 20° ° C. for three days, and are conducted on CTX plates (2% Agar, 5 mM KPO₄ buffer at pH 6, 1 mM CaCl₂), 1 mM MgSO₄) which were purchased pre-poured from LabExpress (Ann Arbor, MI). Urine samples were thawed and diluted at a ratio of 1:2 of urine to CTX buffer (5 mM KPO₄ buffer at pH 6, 1 mM CaCl₂), 1 mM MgSO₄). Urine samples were centrifuged and were then mixed at a ratio of 2:1 diluted urine to 1 M sodium azide. The urine mixture was then spotted at the “plus” marks on each chemotaxis plate. Control buffer mixture was prepared using a 2:1 ratio of CTX buffer to 1 M sodium azide and was spotted at the “minus” marks on each chemotaxis plate. Young adult worms were washed from NGM plates using M9 buffer into conical tubes and were allowed to settle. Worms were then washed three times with CTX buffer to remove traces of the bacterial food source. Approximately 75-100 worms were placed at the center of each plate, which were placed in a 23° C. incubator. After one hour, each plate was removed and placed on a backlight, and an image of each plate was acquired using an iPhone X digital camera.

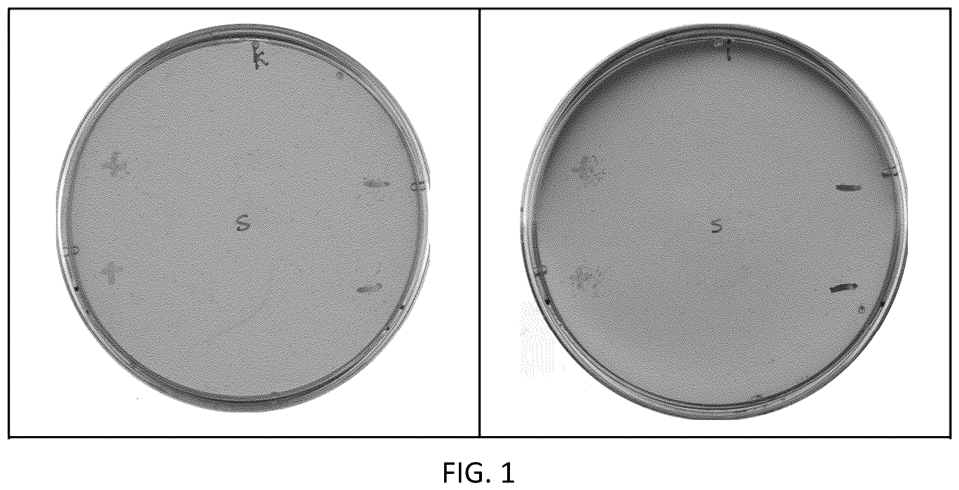

Referring to FIG. 1, chemotaxis was measured by placing C. elegans on the center of a plate. Cancer urine samples were placed on one side of the plate (marked with plus signs). A control plate was created in which C. elegans was placed, but no cancer urine samples. Movement toward the plus signs was measured and deemed positive chemotaxis. That is, it was confirmed that C. elegans orients itself and travels toward the urine sample derived from a companion animal with cancer. FIG. 1 shows two different chemotaxis assay plate images, one without a cancer urine sample, and one with a cancer urine sample on the plus signs. In the control sample, the nematodes dispersed evenly across the plate, and do not congregate towards the plus signs. In contrast, in the cancer sample with a positive chemotaxis index, the nematodes traveled towards the urine sample at the plus signs.

### Data Acquisition

Data was collected by manually counting the worms in each quadrant using Fiji ImageJ software. Replicates are discarded if one of the three conditions are met: (1) if the total for all four quadrants is less than 55 (2); if the highest total quadrant exceeds the sum of the remaining three quadrants; (3) if the quadrant across from the highest total quadrant has fewer than half the animals of any other quadrant. Then, the CI is calculated using the following formula, where Qn is the number of worms in the nth quadrant:

$$CI = \frac{Q 1 - Q 2 + Q 3 - Q 4}{Q 1 + Q 2 + Q 3 + Q 4}$$

A mean CI is calculated from the replicates for each assay that are not discarded.

### Statistical Analysis

Differences between cancer risk groups were assessed using the Welch t-test for data sets with unequal variance. Thresholds for cancer risk assessment were drawn to optimize the accuracy of the data analysis. 95% confidence intervals were specified based on the following formula:

$$C = {t}_{95} * \frac{s}{\sqrt{n}}$$

Where C is the confidence interval, t₉₅ is the t-score, s is the sample standard deviation, and n is the sample size.

### Developing the N.C.S. Study to Distinguish Cancer and Non-Cancer Urine Samples

The N.C.S. Study was developed based on chemotaxis data that was previously generated showing a slight preference of C. elegans for urine samples acquired from cancer patients, as opposed to a slight aversive response to urine from non-cancer patients. The first approach was to determine if the procedure previously used for human cancer detection could be applied to canine urine. While performing chemotaxis, it was found that individual replicate outliers could cause drastic swings in the calculated mean CI. For this reason, replicates that deviated strongly from the other replicates in the assay were discarded. A CI would be discarded if it exhibited a difference greater than 0.25 from the closest other replicate within the assay to reduce the distortion from extreme data points on the mean chemotaxis value. Plates with fewer than 55 worms in the four quadrant boundaries were also discarded, as plates with fewer worms tended to yield a wider range of CI values, leading to greater distortions in the mean. Replicates with unusual distributions were also discarded. Plates were defined as yielding outlier results when the total number of worms in one quadrant exceeded the total number in all three other quadrants combined, or when the number of worms across from the highest total quadrant is less than 50% the total of any other quadrant. These arrangements indicated migration either towards or away from a particular quadrant rather than a particular chemical stimulus and did not provide reliable data for calculating a mean CI.

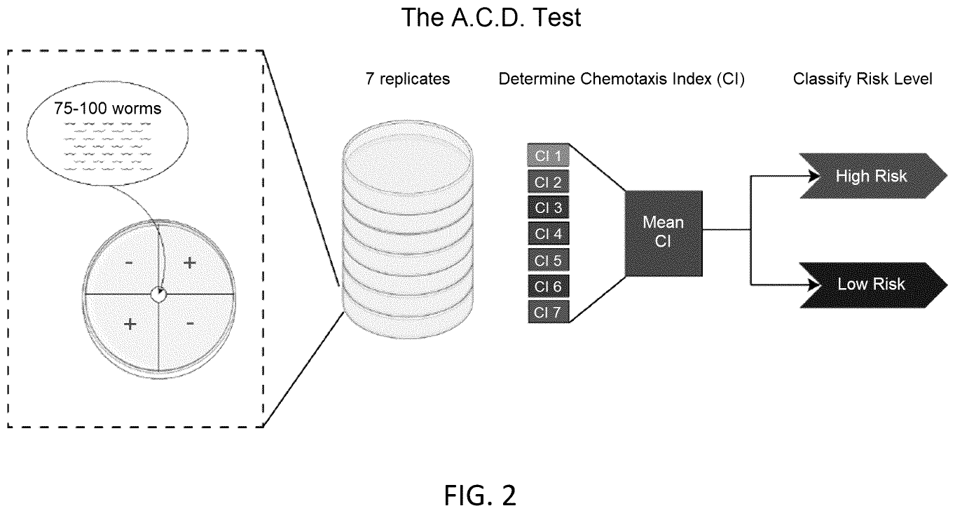

Through these optimizations, the N.C.S. Study was developed to accurately identify urine samples from canine cancer patients (FIG. 2). Previous chemotaxis assays have used anywhere from three to six replicates to determine the mean CI. For canine urine assays, variance was often found in the response and magnitude in individual replicates within an assay. We found that it was necessary to acquire at least five non-discarded replicates for one urine test, four replicates if all are positive or negative. From the CI replicates which were not discarded, the mean CI was calculated, which was then used to assess the level of risk.

### Determining the Level of Cancer Risk for the N.C.S. Study

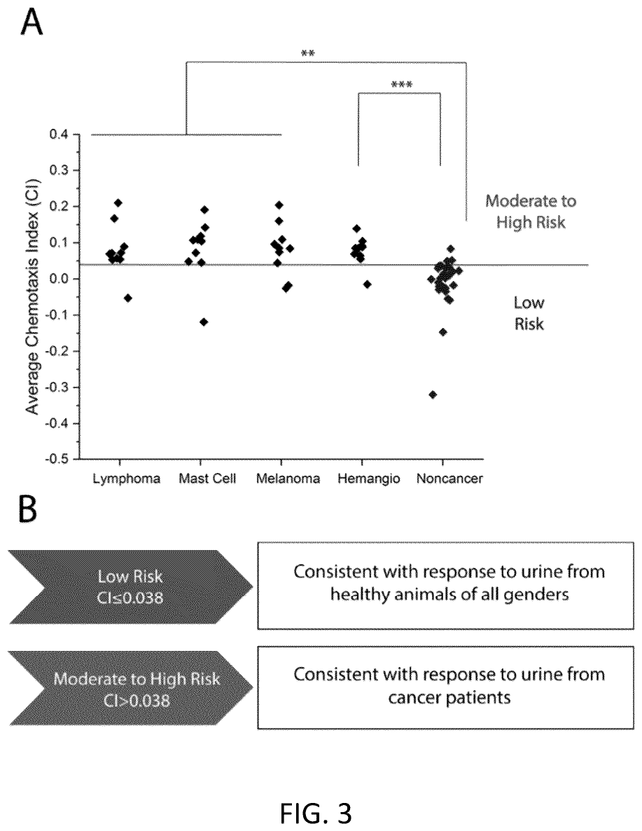

Tests were performed on a series of cancer and non-cancer urine samples to determine if cancer can be detected through positive chemotaxis towards canine urine samples. Assays on a total of eight cancer samples and fourteen non-cancer samples were initially performed. It was found that C. elegans was much more strongly attracted to cancerous urine samples than non-cancer samples (FIG. 3, panel A). From these results, a threshold for elevated cancer risk was set at CI˜0.038, specified by the upper 99.5 percentile determined from a Student t-distribution of the tested non-cancer samples. Mean CI values less than or equal to this value (CI≤˜0.038) were classified as “low risk,” as that is the range of about 85% of non-cancer samples, while results above this value were designated as “moderate to high” (CI>˜0.038) cancer risk. The performance of this assay was assessed by calculating accuracy (fraction of total samples that are correctly classified), sensitivity (fraction of positive samples that are correctly classified), and specificity (fraction of negatives samples that are correctly identified). For this assay, an 88% sensitivity was achieved for cancer detection, and a 93% specificity for correctly classifying non-cancer samples (FIG. 3, panel B). Replicates were also run for a cancer and noncancer sample, which indicated replicable outcomes of the assay risk classification.

### Assessing Detection Rate of Four Common Canine Cancers

To further determine the accuracy of the N.C.S. Study at detecting the presence of cancer, assays were performed on ten additional samples of each of four different types of cancer that are commonly diagnosed in domestic dogs: lymphoma, mast cell tumor, melanoma, and hemangiosarcoma. Additionally, assays were performed on samples from 16 more dogs without a confirmed cancer diagnosis. It was found that all samples yielded a higher mean CI than for non-cancer samples. By combining the data acquired from these forty additional cancer samples and sixteen additional noncancer samples with that in the preliminary data set, we found that the N.C.S. Study yielded a sensitivity of 85% of identifying at least a moderate risk of cancer in each confirmed cancer patient, as compared to the 10% of non-cancer samples identified as at least a moderate cancer risk (Table 1). Overall, by combining all measured CI values for cancer and non-cancer samples, we achieved an accuracy of 87%. These results also showed a statistically significant difference between the mean CI for each type of cancer and the non-cancer samples.

TABLE 1
Data summary of each classification of
cancer versus non-cancer urine samples

| Type | Sample size | Correctly classified | Detection rate |
|---|---|---|---|
| Mast cell tumors | 13 | 12 | 92% |
| Lymphoma | 11 | 10 | 91% |
| Melanoma | 11 | 8 | 73% |
| Hemangiosarcoma | 11 | 9 | 82% |
| Soft-tissue sarcoma | 2 | 2 | 100% |
| Total | 48 | 41 | 85% |
| Non-cancer | 30 | 27 | 90% |

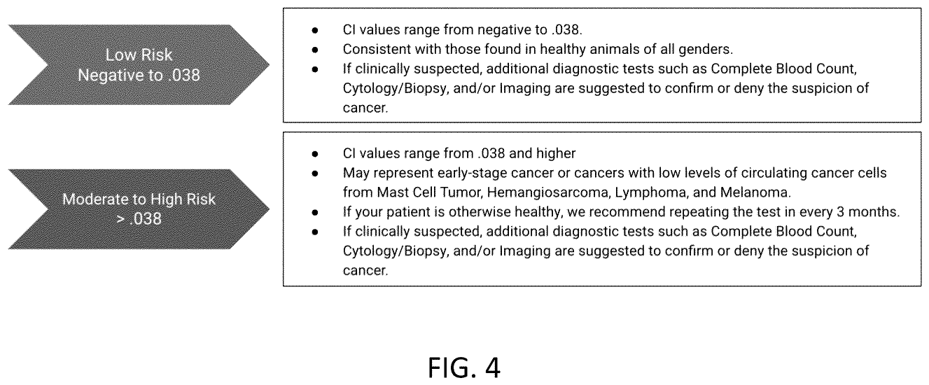

Based on these results, an ability to classify/screen for the cancer risk of canines through C. elegans chemotaxis was demonstrated. An exemplary cancer risk assessment scale, from low to high risk, including recommendations for further diagnostic tests depending upon the cancer risk assessment, is outlined in FIG. 4. Mean CI values less than or equal to a CI≤˜0.038 were classified as “low risk,” and mean CI values >˜0.038 were classified as “moderate to high risk” for cancer. Canine patients from which samples exhibit mean CI values >˜0.1 may further be classified as “high risk” for cancer.

### 2. Early Cancer Detection—Exemplary Six-Step Test

In this example, the early screening test can be executed in six steps:

Step 1. A urine sample contains volatile organic compounds (VOCs) that are released from cancer cells (if cancer is present).

Step 2. The urine sample is diluted to attract or repel olfactory receptors of C. elegans.

Step 3. Measure the capability of C. elegans of preferentially discriminating for cancer-positive urine samples by examining chemotaxis of C. elegans toward cancer-positive urine samples.

Step 4. Chemotaxis index (CI) is calculated based on C. elegans movement toward cancer-positive urine samples.

Step 5. The lab runs multiple statistical analyses to identify cancer risk. The statistical analysis may include analysis of CI, as well as may include additional sample analysis and/or additional subject/patient data, via artificial intelligence (AI) and/or machine learning (ML) to prepare a clinical report of cancer risk in the subject.

Step 6. The clinical report is prepared and sent to the veterinarian indicating whether a cancer risk has been identified for the subject/companion animal.

### 3. Early Cancer Detection—Microfluidic Approach

An exemplary microfluidic approach involves placing the C. elegans in a microfluidic chip with minimal immobilization so the head of the nematode can freely behave and maneuver. The nematode can choose between two separate stimulus streams with different chemicals. In this case, a buffer solution and a urine sample may be employed. This microfluidic method is designed primarily for the collection of behavior data in several forms. These include the percentage of time the head of the nematode resides on the left or the right side of the chip, and mean head angle in the chip (measured with respect to the nematode longitudinal axis of the body in the clamp). Video recordings of behavior can be analyzed in MATLAB using a custom routine to compute head angle in each image. Analysis of behavior may include analysis via AI and/or ML in order to prepare a report of cancer risk for the subject/companion animal.

### 4. On-Chip A.C.D. Test for Screening for/Detecting Cancer in Canines

### The Platform:

An on-plate Animal Cancer Detection (A.C.D.) Test has been developed as a non-invasive method of screening for cancer in canines (see, Namgong et al. (2022) Front. Vet. Sci. 9, 932474), and exemplary risk assessment scales based on this test are outlined in FIG. 3, panel B and FIG. 4. The on-plate A.C.D. The test uses the nematode Caenorhabditis elegans to detect the presence of distinct volatile organic compound (VOC) metabolites in the urine of animals with certain common types of canine cancer. While VOCs associated with the presence of cancer have been studied and tabulated, directly measuring the broad metabolite profile can be challenging. C. elegans is a nematode that is inexpensive to breed and feed, and that possesses a highly sensitive nervous and olfactory system. In conjunction with previous studies conducted on human urine samples, it has been shown that C. elegans is significantly more attracted to cancerous canine urine samples as compared to urine samples obtained from healthy animals. This predictable behavior can be applied towards screening for cancer risk in companion animals. The on-plate A.C.D. Test is conducted on several agar-based chemotaxis plates. While the on-plate A.C.D. Test is effective at differentiating between cancer and non-cancer samples, the on-chip A.C.D. Test of screening for cancer in, for example, canines, and described as follows, performs the analysis more quickly and accurately while reducing the physical waste produced per test. To do this, the first on-chip assay for high-throughput analysis of cancerous VOC content in canine urine samples is developed.

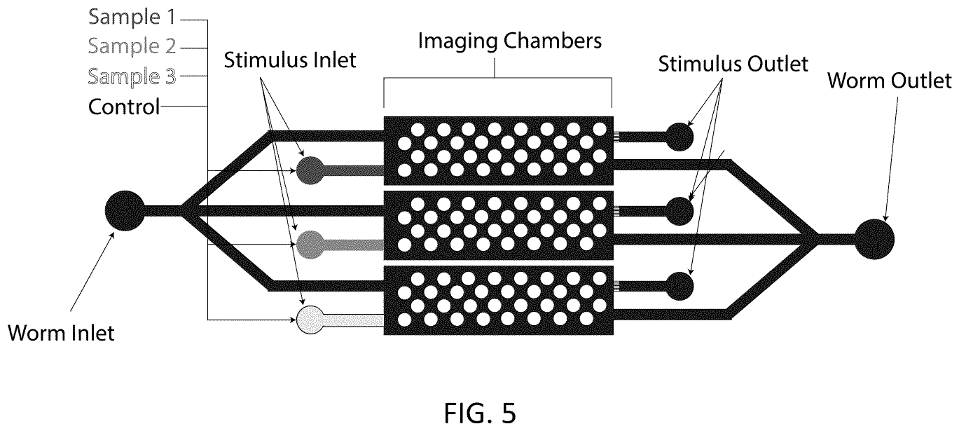

The microfluidic on-chip platform operates through calcium imaging of the AWC neuron, which is the neuron that is primarily responsible for detecting VOCs that are indicative of the presence of cancer (FIG. 5). In a typical test using the on-chip platform, worms are loaded from a single channel into an imaging chamber(s), while each sample to be analyzed is fed into a separate individual imaging chamber. Neuronal response of individual worms to a sample is recorded in the imaging chamber(s). A porous barrier on the stimulus outlet prevents worms from exiting the chamber during assays. Although 3 imaging chambers are depicted in the exemplary on-chip test platform shown in FIG. 5, the number of imaging chambers of the test platform may be greater than 3 or less than 3, e.g., the test platform may have 1 chamber, 2 chambers, or may have 4 chambers, 5 chambers, 6 chambers, 7 chambers, 8 chambers, 9 chambers, 10 chambers, or having even more than 10 chambers. While an on-plate chemotaxis test requires up to an hour to complete, fluorescent calcium gradients of AWC induced by the indicator GCaMP3 can be measured within minutes. Moreover, it has been shown that calcium imaging is a more effective method for the detection of breast cancer from human urine samples. Microfluidic lab-on-a-chip technology has been used for enhancing experimental capabilities in a variety of model organisms, including C. elegans. Polydimethylsiloxane (PDMS) is an exemplary material used for construction of devices that are designed to handle C. elegans, as it is non-toxic, transparent, and gas permeable. Applications of microfluidics towards C. elegans studies include on-chip immobilization and high-resolution imaging. The microfluidic system allows for effective immobilization and neuronal imaging of a fluorescently marked strain of C. elegans indicative of calcium transients. The platform allows for the rapid analysis of urine metabolites. The device is designed to maximize the number of samples that can be assayed in a specified timeframe. It is also optimized to increase the rate of successful operation, and to minimize the modes of operational failure. This device replicates the results that were acquired on-plate indicating a positive response to VOCs corresponding to several types of canine cancer by measuring the neuronal response. This on-chip A.C.D. Test accurately performs rapid cancer screening in a non-invasive manner, and requires less time to perform and generates less physical waste than the on-plate A.C.D. Test.

### Development

Developing a platform for the handling of C. elegans has been challenging for several reasons. The platform needs to be fabricated to handle the rapid loading and unloading of worms from the device. Both PDMS and C. elegans tissue are flexible and deformable, which can lead to challenges in the handling of worms. Additionally, C. elegans is mobile and able to squeeze through small gaps, which can make the trapping or immobilization of worms difficult. For this reason, platform dimensions and inlet flow rates are optimized to balance the smooth locomotion of worms through the channels with effective control of worms within the device. Additionally, the platform needs to be re-usable throughout the course of multiple assays. This is essential for the platform to reduce the time and money required to perform an assay. To do this, a fast and effective clean-up step is implemented, in which all the worms and any traces of previous samples must be purged from the device. Furthermore, the platform must ensure that each urine sample can be efficiently transferred to the platform, and that the chip and inlet tubing are sufficiently washed to remove any odors/contamination from previous samples. Development of the device includes determining the most common modes of operational failure and incorporating fail-safes into the design to ensure the continuous operation of the platform. Finally, the platform ensures accurate and efficient acquisition of quantitative calcium imaging data. A fluorescent inverted microscope customized to acquire repeat images of GCaMP signaling is used to acquire fluorescent calcium images of the AWC neuron. The customized setup is optimized to acquire images at the correct resolution and intensity. Additionally, an automated image analysis algorithm to quantify the intensity of the calcium gradient is provided. This is achieved through filtering and binarization techniques using the MATLAB Image Processing Toolbox. A set of image ground truths is manually created to measure the accuracy of the algorithm.

### Optimization

After development and efficient operation, a high level of performance is shown by validating the test on a set of urine samples from healthy animals and cancer patients. The objective is to achieve a sensitivity and specificity that is comparable or better than those yielded from the on-plate experiments. It has been found that on-plate assays were highly sensitive to their environment. Performing assays on-chip introduce additional environmental factors that can influence the chemosensory response to the stimuli. Most notably, the process of loading animals onto the chip does not cause any stress that would influence odor response needs to be ensured, as has been previously observed in responses to other environmental stressors. The concentration of urine in solvent buffer is also optimized. It was found that a 1:2 ratio of urine sample to control buffer produced the strongest response differential between cancer and non-cancer samples in the on-plate A.C.D. test. It is ensured that enough urine will be acquired to run repeat experiments if necessary. Each assay requires no more than about 500-750 μL of urine to run. The plate-based method typically needs at least 75-100 C. elegans for each assay, and typically takes 2 hours to draw any conclusion. The on-chip assay for high-throughput analysis of cancerous VOC content in canine urine samples is operated with the minimum number of C. elegans required to draw significant data with high-accuracy results.

### 5. On-Chip A.C.D. Test Protocol

### Typical Protocol for Operation of On-Chip A.C.D. Platform:

The following is an outline for an exemplary test protocol performed by an exemplary system for a high-throughput microfluidic screening for cancer in patient-derived urine samples. The analysis, in addition to behavioral response, chemotaxis response, and/or neuronal response quantitation, may include uranalysis and GC/MS analysis of the urine sample, as well as additional patient data, to arrive at a cancer risk assessment, a determination of a probability/likelihood that the patient has cancer, and/or a determination whether the patient may be afflicted with a cancer.

1. Grow C. elegans on Nematode Growth Media (NGM) agar plates seeded with lawns of the HB101 strain of E. coli bacteria for about three days after embryos hatch until adulthood.

2. Load 1000 L of urine dissolved in buffer into each sample tube. Place each tube into the sample cartridge, noting the order in which they are loaded.

3. Specify the number of samples to be assessed, and the name of each sample to be assessed in the order of loading in the operational GUI for the platform.

4. Wash worms from the plates using M9 worm handling buffer, and place in a microtube. Wash, e.g., three times, with odor-neutral control (CTX) buffer.

5. Load about 50 worms per urine sample to be tested into the worm injection tube.

6. Initialize the pre-assay flush cycle on the GUI for the platform. Using an automated flow pump, flush the microfluidic imaging chip with odor-neutral control buffer to ensure all odorants are purged from the chambers.

7. Initialize worm loading using the GUI for the platform. Pump the worms from the injection tube into the filtration chamber.

8. Filter eggs and larvae from the worm population using the filtration features in the chamber.

9. Begin feeding each worm from the filtration chamber into the length analysis chamber. Assess the length and width of each individual animal to determine which are below a length and width indicative of young adult, non-gravid (non-egg-laying) worms. Feed about 20 worms into each analysis chamber.

10. Acquire images of baseline fluorescent AWC neuronal calcium activity of worms in control buffer.

11. Initiate urine assays using the control GUI for the platform.

12. Begin pumping control buffer, e.g., for about three minutes into the chambers.

13. Sequentially pump urine sample dissolved in control buffer to each chamber, being mixed in a 1:100 ratio with the control buffer. Acquire intermittent neuronal activity images about 5 seconds before through 60 seconds after each sample is added.

14. Simultaneously, add about 100 μL urine sample dropwise to each square on a urinalysis strip. Each urine strip is sent to a color analyzer where the strip parameters are quantified.

15. Send remaining urine to a GC/MS chamber for GC/MS analysis.

16. Once urinalysis data is acquired, the automated flow pump is activated, and each valve is sequentially opened, allowing for control buffer to flow into each chamber sequentially. Intermittent neuronal activity images are acquired about 30 seconds before and about 3 minutes after control buffer flows into chamber.

17. Acquired images are saved sequentially with timestamps to a folder corresponding to each sample.

18. Worms are flushed from the device through the worm outlet. A diluted mixture of bleach and sodium hydroxide (NaOH) is pumped into each chamber and allowed to sit for ten minutes, which sterilizes the chamber and degrades the corpses of any remaining worms. Control buffer is flowed through the chambers to flush the bleach/NaOH solution as well as any remaining worm debris from the chambers.

19. Using an AI-based image-analysis software, the intensity of calcium activity is quantified at each time point. The software automatically identifies the location of each fluorescent neuron without need for user interference and tracks the motion of each worm over time. The mean intensity of the neuron is assessed at each time point and plotted to determine the magnitude of the response.

20. Mean calcium activity and urinalysis data for all assessed samples are compiled.

21. Using the combination of C. elegans AWC neuronal calcium response, urinalysis data, and GC/MS data, along with the age, sex, and medical history of the subject, an AI algorithm is used to assess the probability that the patient has cancer.

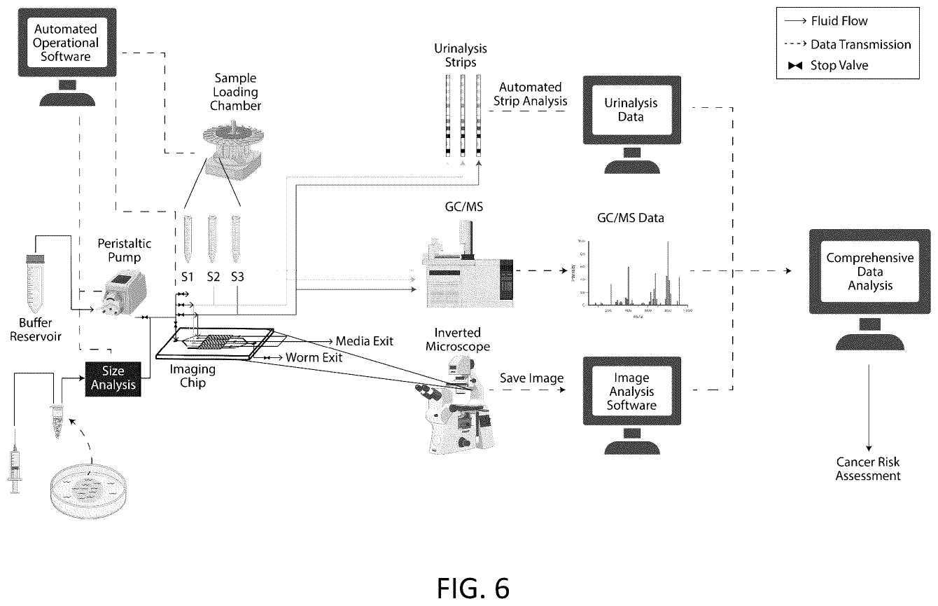

A flowchart illustrating the system and included components to carry out the on-chip A.C.D. protocol described above, including worm (C. elegans) behavioral, chemotaxis, and/or neuronal response analysis, uranalysis, and GC/MS analysis of a subject/patient-derived sample, is depicted in FIG. 6.

Although the foregoing subject matter has been described in some detail by way of illustration and example for purposes of clarity of understanding, it will be understood by those skilled in the art that certain changes and modifications can be practiced within the scope of the appended claims.

1. A method of screening for cancer in a subject, the method comprising:
detecting a behavioral response, a chemotaxis response, and/or a neuronal response of a nematode, or nematodes, to a biological sample obtained from the subject;
determining whether the behavioral response, chemotaxis response, and/or neuronal response of the nematode or nematodes exceeds or is within a threshold for a positive response, wherein a positive response to the sample indicates the subject has cancer or is at risk for having cancer; and
identifying the subject as having cancer or as being at risk for having cancer if the behavioral response, chemotaxis response, and/or neuronal response of the nematode or nematodes exceeds or is within a threshold for a positive response.

2. The method of claim 1, wherein the nematode, or nematodes, is a wild-type nematode, a mutant nematode or a transgenic nematode.

3. The method of claim 1, wherein the nematode, or nematodes, is a wild-type recombinant, or transgenic Caenorhabditis elegans (C. elegans).

4. (canceled)

5. The method of claim 1, wherein a positive chemotaxis response comprises chemotaxis toward the biological sample.

6. The method of claim 1, wherein the biological sample is urine.

7. The method of claim 1, wherein the cancer is selected from the group consisting of hemangioma, lymphoma, osteosarcoma, plasma cell tumor, and mast cell tumor, transitional cell carcinoma and/or melanoma.

8. A method for determining the effect of a treatment on the development of cancer in a subject, the method comprising:
detecting a behavioral response, a chemotaxis response, and/or a neuronal response of a nematode, or nematodes, to a biological sample obtained from the subject;
determining whether the behavioral response, chemotaxis response, and/or neuronal response exceeds or is within a threshold for a positive response, wherein a positive response to the sample indicates the treatment has not rendered the cancer undetectable; and
identifying the treatment of the subject as not having rendered the cancer undetectable if the behavioral response, chemotaxis response, and/or neuronal response exceeds or is within a threshold for a positive response.

9. The method of claim 8, wherein a positive chemotaxis response comprises chemotaxis toward the biological sample.

10. The method of claim 1, wherein the subject is a companion animal.

11. A method of screening for and/or detecting cancer in a subject, the method comprising:
detecting a behavioral response, a chemotaxis response, and/or a neuronal response of a nematode, or nematodes, to (a) a biological sample obtained from a subject, and (b) at least one sample from the same or a different source, using a microfluidic approach to detect the behavioral response, the chemotaxis response, and/or the neuronal response;
determining the behavioral response, chemotaxis response, and/or the neuronal response of the nematode or nematodes, wherein if the behavioral response, chemotaxis response, and/or the neuronal response of the nematode or nematodes exceeds or is within a pre-determined response to the samples indicate the subject has cancer or is at risk for having cancer; and
identifying the subject as having cancer or as being at risk for having cancer if the behavioral response, chemotaxis response, and/or the neuronal response of the nematode or nematodes exceeds or is within the pre-determined threshold response to the samples.

12. The method of claim 11, wherein the nematode is a wild-type nematode, a mutant nematode or a transgenic nematode.

13. The method of claim 11, wherein the nematode, or nematodes, is C. elegans.

14. The method of claim 11, wherein a positive chemotaxis response comprises chemotaxis toward the biological sample.

15. The method of claim 11, wherein the biological sample is urine.

16. (canceled)

17. A kit for: (i) determining a presence of, and/or a risk of a presence of a cancer in a subject; or (ii) determining the effect of a treatment on development of cancer in a subject wherein the kit comprises:
(a) a receptable to contain a biological sample from the subject; and
(b) instructions providing guidance regarding the biological sample and steps to obtain results from a test to determine a behavioral response, a chemotaxis response, and/or a neuronal response of a nematode to the biological sample.

18. The kit of claim 17, wherein the subject is a companion animal or a human subject:

19. A computer-implemented method executed on a computer system, or computer readable medium operated thereon for: (i) determining a presence of, and/or a risk of a presence of a cancer in a subject; or (ii) determining an effect of a treatment on cancer development in subject, the method comprising:
a) monitoring a behavioral response, a chemotaxis response, and/or a neuronal response for a nematode, or a plurality of nematodes, to a biological sample;
b) determining whether the behavioral response, the chemotaxis response and/or the neuronal response exceeds or is within a threshold for a positive response; and
c) identifying whether there is a presence of and/or a risk for a presence of cancer in the subject,
wherein the presence of, and/or the risk of a presence of cancer in the subject is determined if the behavioral response, the chemotaxis response or the neuronal response exceeds or is within the threshold for a positive response.

20. The computer-implemented method of claim 19, wherein determining if the behavioral response, the chemotaxis response or the neuronal response exceeds or is within a threshold for a positive response comprises evaluation of the behavioral response, the chemotaxis response or the neuronal response of the nematode or plurality of nematodes via artificial intelligence and/or machine learning to determine if the behavioral response, the chemotaxis response or the neuronal response exceeds or is within the threshold for a positive response.

21. (canceled)
